(19) United States
(12) Patent Application Publication
(10) Pub. No.: US 2026/0327675 A1
(43) Pub. Date: Oct. 22, 2026

(54) FLUOROMETRIC-BASED COUPLED ENZYME SYSTEMS

(71) Applicant: UNIVERSITY OF SOUTH CAROLINA, COLUMBIA, SC (US)

(72) Inventor: EDWARD L. D'ANTONIO, COLUMBIA, SC (US)

(21) Appl. No.: 19/650,786

(22) Filed: Apr. 17, 2026

Related U.S. Application Data
(60) Provisional application No. 63/790,854, filed on Apr. 18, 2025.

(51) Int. Cl. C12Q 1/34; C12Q 1/32; C12Q 1/58

(52) U.S. Cl. CPC C12Q 1/34; C12Q 1/32; C12Q 1/58; C12Y 104/01002; C12Y 305/01005; C12Y 305/03001

(57) ABSTRACT

In general, disclosed herein are methods of screening for an arginase inhibitor. The method may include contacting a first substrate with a sample comprising a first enzyme comprising arginase and a candidate arginase inhibitor to form urea; reacting the urea with a second enzyme to form ammonia; reacting the ammonia with a third enzyme, a substrate of the third enzyme, and a fluorophore; and measuring a fluorescence signal of the sample, wherein a decrease in the fluorescence signal of the sample corresponds to an arginase inhibitor compared to a control sample lacking the candidate arginase inhibitor.

### CROSS REFERENCE TO RELATED APPLICATIONS

This application claims the filing benefit of U.S. Provisional Patent Application Ser. No. 63/790,854, filed on Apr. 18, 2025, which is incorporated herein by reference.

### BACKGROUND

Arginase (EC 3.5.3.1) is a manganese-dependent metalloenzyme that catalyzes the hydrolytic cleavage of the guanidinium side chain of L-arginine to yield L-ornithine and urea as the stoichiometric products of the reaction. Two genetically distinct isoforms are recognized in mammals: arginase-1 (ARG1), a cytosolic enzyme predominantly expressed in hepatocytes and implicated in the urea cycle, and arginase-2 (ARG2), a mitochondrial enzyme expressed in kidney, prostate, and other extrahepatic tissues. Both isoforms share a conserved binuclear manganese cluster at the active site and are subject to allosteric regulation.

Arginase activity is of substantial biological and therapeutic relevance. ARG1 is a validated driver of immunosuppression within the tumor microenvironment, where its overexpression in tumor-associated macrophages and myeloid-derived suppressor cells depletes extracellular L-arginine, thereby attenuating T-cell receptor signaling and the proliferative capacity of cytotoxic lymphocytes. ARG2 has been implicated in vascular dysfunction, pulmonary arterial hypertension, and diabetic complications through competition with endothelial nitric oxide synthase (eNOS) for their shared substrate L-arginine. Accordingly, arginase inhibitors represent an active area of therapeutic investigation, and robust, quantitative, high-throughput-compatible arginase activity assays are essential tools for inhibitor discovery and pharmacological characterization.

Existing methods for detecting arginase activity suffer from well-recognized limitations. Colorimetric urea-based endpoint assays, including the diacetyl monoxime and Berthelot methods, provide only a single terminal measurement, preclude kinetic characterization of inhibitor mechanism, and are susceptible to interference from chromogenic compounds present in complex biological matrices. Radiometric assays employing ¹⁴C- or ³H-labeled L-arginine, while sensitive, require radioactive handling infrastructure, generate radiological waste, and are incompatible with high-throughput screening formats. Antibody-based detection of L-ornithine or urea lacks the sensitivity and dynamic range necessary for low-abundance enzyme preparations.

As such, what is needed in the art are coupled enzyme systems that transduce arginase-mediated L-arginine hydrolysis into a fluorescent signal through a sequence of operably coupled enzymatic reactions, enabling continuous kinetic measurement with sensitivity, dynamic range, and throughput characteristics superior to conventional methods.

### SUMMARY

In general, disclosed herein are methods of screening for an arginase inhibitor. The method may include contacting a first substrate with a sample comprising a first enzyme comprising arginase and a candidate arginase inhibitor to form urea; reacting the urea with a second enzyme to form ammonia; reacting the ammonia with a third enzyme, a substrate of the third enzyme, and a fluorophore; and measuring a fluorescence signal of the sample, wherein a decrease in the fluorescence signal of the sample corresponds to an arginase inhibitor compared to a control sample lacking the candidate arginase inhibitor.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates the chemical reaction, reagents, products, and enzymes of the fluorometric arginase assay.

FIG. 2A depicts SDS-PAGE with lane 1 as the molecular weight marker; lane 2 as human arginase I (HAI); lane 3 as CeURE; and lane 4 as BtGDH. This gel shows the purity of the enzymes used in the assay reaction.

FIG. 2B depicts the Michaelis-Menten plot obtained when using human arginase I and substrate L-arginine (N=3). The K_{M} value was determined to be 0.04417±0.004245 mM at pH 8.5.

FIG. 2C depicts the Lineweaver-Burk plot observed for different concentrations of the reference inhibitor 2(S)-amino-6-boronohexanoic acid (ABH) vs. HAI (N=3). The inhibition constant (Kᵢ) value was determined to be 12.0±4.2 μM, indicating strong inhibition of HAI.

FIG. 3A depicts the SDS-PAGE with lane 1 as the molecular weight marker; lanes 2-4 as His-tagged Leishmania infantum arginase (LiARG(K13-L329)) or simply Δ12-LiArg.

FIG. 3B depicts the Michaelis-Menten plot obtained when using Δ12-LiArg and substrate L-arginine (N=3). The K_{M} value was determined to be 2.466±0.3802 mM at pH 8.5.

FIG. 3C depicts the Lineweaver-Burk plot observed for different concentrations of the reference inhibitor ABH vs. Δ12-LiArg (N=2). The Kᵢ value was determined to be 23.3±9.4 μM.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure covers such modifications and variations as come within the scope of the appended claims and their equivalents.

In general, the present disclosure is directed to high-throughput, fluorometric assay systems and methods for using the systems to detect arginase activity. Advantageously, methods disclosed herein can be utilized for high-throughput screening in therapeutic drug discovery applications. For instance, systems and methods disclosed herein may be useful for high-throughput screening of potential arginase inhibitors.

As used herein, “arginase activity” refers to the catalytic rate of hydrolysis of L-arginine to L-ornithine and urea by arginase, expressed in units of nanomoles of L-arginine hydrolyzed per minute (nmol/min) or as inhibition constants derived therefrom under defined assay conditions.

The high-throughput, fluorometric-based assays disclosed herein may include an enzyme-coupled system, which is predicated on the stoichiometric relationship between arginase-catalyzed L-arginine hydrolysis and a measurable change in fluorescence intensity through a sequence of operably coupled enzymatic reactions. As used herein, an “enzyme-coupled system” refers to a multi-enzyme cascade in which the product of one enzymatic reaction serves as the substrate for a subsequent enzymatic reaction, such that the overall system transduces the activity of a primary enzyme of interest into a detectable signal generated by a terminal reporting reaction. The enzyme cascade may be designed such that each downstream enzymatic step proceeds at a rate that is non-rate-limiting relative to the primary arginase reaction under assay conditions disclosed herein, ensuring that the fluorescent signal change observed at the terminal reporting step reflects the kinetics of arginase-mediated L-arginine hydrolysis.

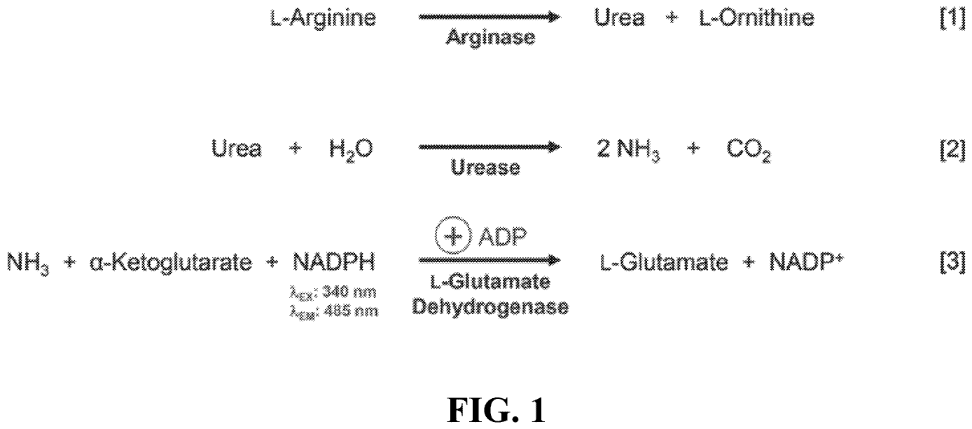

In example embodiments, the high-throughput, fluorometric-based assays disclosed herein may be utilized to determine the efficacy of a candidate arginase inhibitor. FIG. 1 provides a general design for a high-throughput, fluorometric assay that may be used to examine a candidate inhibitor of arginase.

In example embodiments, the high-throughput, fluorometric-based assay system may include a first enzyme and a substrate of the first enzyme.

In example embodiments, the first enzyme may be arginase. For instance, arginase may be arginase I. For instance, arginase I may include, but is not limited to, human arginase I or parasite-derived arginase I. In one example embodiment, arginase I may be human arginase I. In another example embodiment, arginase I may be parasite-derived arginase I, such as Leishmania infantum arginase I.

In example embodiments, the substrate of the first enzyme may be L-arginine.

While disclosed assay reagents can be particularly beneficial for the discovery of arginase inhibitors, potential inhibitors of other enzyme/substrate interactions can also be examined using disclosed assays. As is the case with arginase, when screening other potential inhibitors, the high-throughput, fluorometric-based assay system may include the candidate enzyme inhibitor, the enzyme of interest, and a substrate for the enzyme of interest. For instance, a substrate can include, without limitation, a polynucleotide (e.g., a ssDNA, a ssRNA, a dsDNA, a dsRNA), a polypeptide (e.g., a protein substrate or a fragment thereof that includes a recognition motif for the enzyme), a carbohydrate, a glycoprotein, etc.

In example embodiments, the high-throughput, fluorometric-based assay system may include a second enzyme. For instance, the second enzyme may include, but is not limited to, urease, carbonic anhydrase, nitrate reductase, nitrite reductase, asparaginase, protease, adenosine deaminase, or a combination thereof. In one example embodiment, the second enzyme may be urease. For instance, urease may be Canavalia ensiformis urease.

In example embodiments, the high-throughput, fluorometric-based assay system may include a fluorophore. For instance, the fluorophore may be an electron transfer mediator. In general, electron transfer mediator may include, and without limitation to, nicotinamide adenine dinucleotide (NAD⁺), NADH, nicotinamide adenine dinucleotide phosphate (NADP+), NADPH, flavin adenine dinucleotide (FAD), FADH₂, flavin mononucleotide (FMN), FMNH₂, Coenzyme A, Coenzyme Q, tryptophan tryptophylquinone (TTQ), pyrroloquinolinequinone (PQQ), adenosine triphosphate (ATP), guanosine triphosphate (GTP), cytidine triphosphate (CTP), thymidine triphosphate (TTP), uridine triphosphate (UTP), adenosine diphosphate (ADP), guanosine diphosphate (GDP), cytidine diphosphate (CDP), thymidine diphosphate (TDP), and uridine diphosphate (UDP), or any combination thereof. Preferred electron transfer mediators can depend on the specific characteristics of an assay, as will be understood by one skilled in the art. In one example embodiment, the fluorophore may be NADPH.

In example embodiments, the high-throughput, fluorometric-based assay system may include one or more supporting enzymes. The supporting enzyme(s) can be from any source. For instance, the supporting enzyme may include, but is not limited to, glutamate dehydrogenase, α-ketoglutarate, lactate dehydrogenase, pyruvate dehydrogenase, fructose dehydrogenase, sucrose dehydrogenase, glucose dehydrogenase, glucose-6-phosphate dehydrogenase, glyceraldehyde-3-phosphate dehydrogenase, sorbitol dehydrogenase, isocitrate dehydrogenase, alpha-ketoglutarate dehydrogenase, succinate dehydrogenase, malate dehydrogenase, yellow enzyme, glutamate dehydrogenase, glycerol 1-phosphate dehydrogenase, or a combination thereof.

In one example embodiment, the supporting enzyme may be glutamate dehydrogenase. For instance, glutamate dehydrogenase may be Bos taurus glutamate dehydrogenase.

In another example embodiment, the supporting enzymes may be glutamate dehydrogenase and α-ketoglutarate. It is understood that glutamate dehydrogenase reversibly converts α-ketoglutarate to glutamate.

The high-throughput, fluorometric-based assay can include additional materials as are generally known in the art. By way of example, in those embodiments in which the enzyme of interest and/or one or more supporting enzymes is a metalloenzyme that utilizes a metal ion as a cofactor, the assay can include a salt of the metal, e.g., a salt of iron, copper, magnesium, zinc, manganese, calcium, etc., or any combination thereof.

In one example embodiment, the high-throughput, fluorometric-based assay can include a buffer, for instance, to ensure desired activity of the enzyme of interest, to ensure solubility of one or more components of the assay, to prevent degradation of enzyme activity, to encourage product purification, and the like. Exemplary buffers can include, without limitation, phosphate buffers, Tris-HCl, triethanolamine (TEA), 4-(2-hydroxyethyl)piperazine-1-ethanesulfonic acid (HEPES), 4-morpholinepropanesulfonic acid (MOPS), etc., and combinations thereof.

In one example embodiment, a high-throughput, fluorometric-based assay system can be provided as a combination kit. As used herein, the terms “combination kit” or “kit” is intended to refer to one or more components of an assay mixture and/or any additional components that can be used to package, sell, market, deliver, and/or provide the assay components to a user. Such additional components include, but are not limited to, packaging, syringes, pipettes, blister packages, and the like. When one or more of the compounds of an assay are provided simultaneously in a kit, the combination kit can contain the compounds in a single package. When one or more of the compounds are not provided simultaneously, the combination kit can contain each component in any combination. The separate kit components can be contained in a single package or in separate packages within a single kit package.

By way of example, a kit can include the primary enzyme of interest (e.g., arginase), a substrate for the enzyme, and a fluorophore probe as described. In example embodiments, the kit can also include one or more supporting enzymes, one or more cofactors, buffers, etc. In some example embodiments, a kit can include only a portion of the components of an assay. For instance, an assay kit may be provided that does not include one or more of the electron transfer mediator(s), cofactors, buffers, etc., though these components may be utilized in carrying out an assay protocol.

In some example embodiments, a kit also includes instructions printed on or otherwise contained in a tangible medium of expression. The instructions can provide information regarding the content of the kit, safety information regarding components of the kit, information regarding the assay, indications for successful use of the kit, and/or instructions for carrying out an assay. In some embodiments, the instructions can provide directions and protocols for providing the assay.

To carry out an assay, an assay composition can be formed that includes the enzyme of interest, the substrate, the candidate inhibitor, and any supporting materials as necessary, e.g., supporting enzymes, cofactors, buffers, etc.

In example embodiments, methods disclosed herein may be useful for high-throughput screening of potential inhibitors. For instance, detecting arginase activity may include contacting a sample with a high-throughput, fluorometric assay system. In one example embodiment, the sample may include a first enzyme and a candidate inhibitor compound. Subsequently, the sample may be contacted with a first substrate. Next, the sample may be contacted with a second enzyme. Further, the sample may be contacted with one or more additional enzymes (e.g., a supporting enzyme) and a fluorophore probe.

For instance, turning again to FIG. 1, to detect arginase activity, the first step of the high-throughput, fluorometric-based assay system may involve the chemical reaction between the substrate L-arginine and the enzyme arginase [1]. Arginase converts L-arginine to urea and L-ornithine; urea then reacts with urease and water to produce ammonia and a by-product, CO₂ [2]. The next step involves the ammonia made from the urease reaction and combines it with the reagent α-ketoglutarate, in the presence of NADPH (an intense fluorophore). The third enzyme, Bos taurus glutamate dehydrogenase (BtGDH), converts those substrates to form L-glutamate and NADP+, thereby signaling that our reaction has occurred, as we observe a loss in fluorescence [3]. NADP⁺ is a fluorometrically silent compound, whereas NADPH is an intense fluorophore, and that is the basis for why a decrease in fluorescence occurs in this assay. Urea and L-ornithine are also fluorometrically silent, so no assay can measure the rate at which those products are produced using high-throughput screening.

NADPH possesses intrinsic fluorescence in its reduced form, with excitation maximum at approximately 340 nm and emission maximum at approximately 460 nm, arising from the reduced nicotinamide ring of the adenine dinucleotide. Upon oxidation to NADP⁺ by glutamate dehydrogenase, the nicotinamide ring is converted to its oxidized, non-fluorescent form, producing a stoichiometric decrease in fluorescence emission that is continuously monitored as the assay output signal.

The high-throughput screening method disclosed herein may enable researchers to screen thousands of compounds using a fluorescence-based microplate reader to inhibit enzymes such as arginase. In this assay, having a decrease in fluorescence requires adjustment in the calculations for the relative fluorescence unit (RFU). This calculation may be established through the adjustment in RFU (RFU_{ADJ}). RFU_{ADJ}=(ΔRFU_{NADPH}−ΔRFU_{Sample}), which is proportional to the rate gain of [NADP⁺]. Stoichiometry must be taken into account, as 1.0 mole of L-arginine consumed in the arginase reaction ultimately produces 2.0 moles of NADP⁺ (FIG. 1). Finally, if desired, Michaelis-Menten plots regarding an RFU_{ADJ} on the y-axis is proportional to rate of NADP⁺ production during a reaction time optimum (tₒₚₜ) of 5.0 min at 22° C. for human arginase I, for example. Thus, RFU_{ADJ} on the y-axis may also be written as RFU_{ADJ}/At (min-) with units of inverse minutes, as a form of rate.

In example embodiments, arginase assays disclosed herein may be useful to perform studies to screen malvidin analogs against LiArg in hopes of finding one that exhibits strong inhibition, for it can potentially be utilized as a therapeutic against leishmaniasis.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Example 1

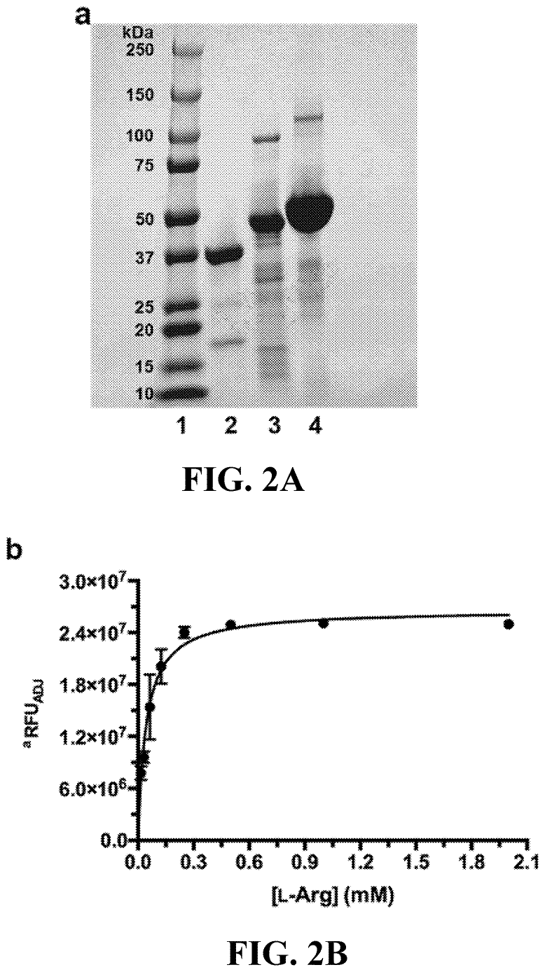

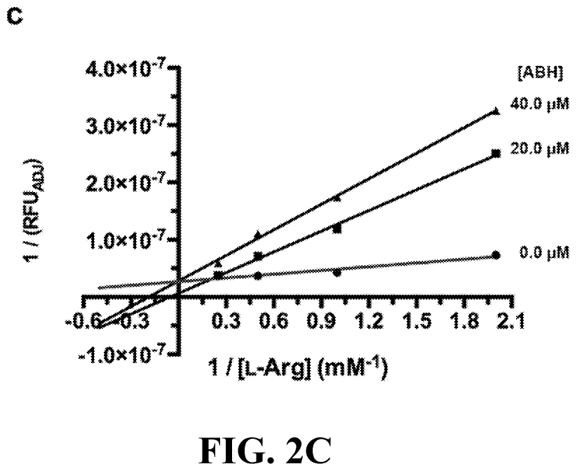

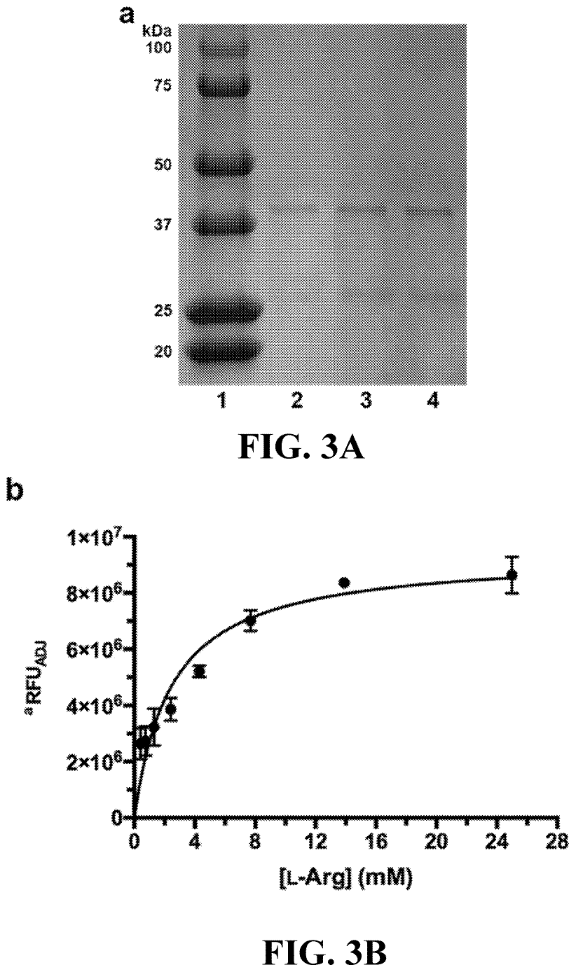

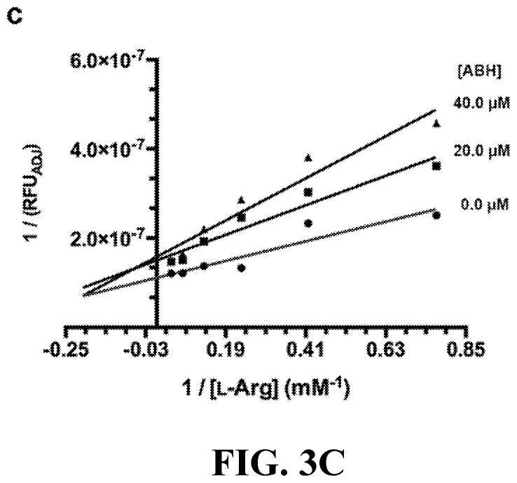

To measure the activity of arginase, human arginase I (HAI) was utilized. The assay disclosed herein employs the use of an enzyme-coupled reaction involving Bos taurus glutamate dehydrogenase (BtGDH) and Canavalia ensiformis urease (CeURE). It follows a 3-enzyme catalyzed reaction sequence that ultimately leads to the conversion of NADPH into NADP⁺. Once NADP⁺ was produced, the assay loses fluorescence, and the signal loss is measured by a BMG VANTAStar multimode microplate reader. FIGS. 2A and 3A indicate the purity of the samples used in the assays that were performed. Table 1 shows relevant properties of the same enzymes.

The final assay concentrations (F.A.C.) for each component were:

| | F.A.C. (μg/mL) |
|---|---|
| CeURE | 100.0 |
| BtGDH | 6.3 |
| Arginase (HAI or LiARG) | 12.5 |

TABLE 1
Properties of enzymes involved in the fluorometric arginase assay.

| | ^{a}GenBank | ^{b}Molecular | No. of |
|---|---|---|---|
| Enzyme | Accession No. | Mass (Da) | Amino Acids |
| HAI | 75766132 | 34,737 | 322 |
| His-tagged Δ12-LiArg | CAC9543944.1 | 36,053 | 330 |
| ^{c}Ce URE | AAA83831.1 | 90,765 | 840 |
| ^{c}Bt GDH | 13096305 | 55,561 | 501 |

^{a}https://www. [symbol] .gov/genbank/
^{b}Molecular mass for the [symbol] subunit.
^{c}Purchased from Worthington Biochemical Corporation (Lakewood, NJ) without further purification. [symbol]
indicates data missing or illegible when filed

For HAI and L-arginine, the K_{M}=0.04417±0.004245 mM and for Δ12-LiARG and L-arginine, the K_{M}=2.466±0.3802 mM, respectively (FIGS. 2B and 3B). The following Kᵢ values from Lineweaver-Burk plots in FIGS. 2C and 3C are reported as 12.0±4.2 μM for HAI and 2(S)-amino-6-boronohexanoic acid (ABH) (N=3) and 23.3±9.4 μM for Δ12-LiARG and ABH (N=2).

The K_{M} value for HAI/L-arginine closely matched the reported value by Di Costanzo et al., which was 0.08 mM at a pH of 8.5, and utilizing the assays disclosed herein the K_{M} value for HAI/L-arginine was measured as 0.04417±0.004245 mM at a pH of 8.5. This finding showcases a tight correlation with interlaboratory precision. The temperature of this reaction occurred at was 22° C. for 5 minutes. In addition, as reported by Gracia et al., a value for the K_{M} of LiArg and L-arginine was 5.1±1.1 which is very similar to results obtained from the reactions with Δ12-LiARG herein, which shows it to be 2.466±0.3802 mM (performed at 37° C., 5 minutes reaction time, and at a pH of 8.5.

The results for both reactions with HAI and Δ12-LiARG suggest that the assay is a success and can produce replicable results that are in accordance with other parameter values from other research groups. The Kᵢ values also suggest that there is inhibition, and the strength of the inhibition as well is strong. It displays that our assay is not reacting with any other components in the reaction mixture. A confirmatory assay using ABH, CeURE, and BtGDH was performed and a normal percent inhibition (NPI) value of less than 20% was obtained, such as NPI=0.211±0.097% (N=8), when a final assay concentration of ABH of 50.0 μM was implemented. This reference inhibitor ABH was not interfering with the other enzymes involved in the reaction. Since we measured the decrease of fluorescence, NPI was accordingly adjusted to a modified version, as follows:

$${NPI}_{Modified} = \frac{{x}_{i} - {c}_{+}}{{c}_{-} - {c}_{+}} \times 100 \% ,$$

where xᵢ is the measurement of the i^{th} compound, c₊ and c₋ are the means of measurement from the positive and negative controls respectively, in the confirmatory assay.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A high-throughput coupled enzyme method of screening for an arginase inhibitor, the method comprising:
contacting a first substrate with a sample comprising a first enzyme comprising arginase and a candidate arginase inhibitor to form urea;
reacting the urea with a second enzyme to form ammonia;
reacting the ammonia with a third enzyme, a substrate of the third enzyme, and a fluorophore; and
measuring a fluorescence signal of the sample, wherein a decrease in the fluorescence signal of the sample corresponds to an arginase inhibitor compared to a control sample lacking the candidate arginase inhibitor.

2. The method of claim 1, wherein the first enzyme is arginase I.

3. The method of claim 1, wherein the first substrate is L-arginine.

4. The method of claim 1, wherein the second enzyme is selected from a group consisting of urease, carbonic anhydrase, nitrate reductase, nitrite reductase, asparaginase, protease, adenosine deaminase, or a combination thereof.

5. The method of claim 4, wherein the second enzyme is urease.

6. The method of claim 1, wherein the third enzyme is selected from a group consisting of glutamate dehydrogenase, lactate dehydrogenase, pyruvate dehydrogenase, fructose dehydrogenase, sucrose dehydrogenase, glucose dehydrogenase, glucose-6-phosphate dehydrogenase, glyceraldehyde-3-phosphate dehydrogenase, sorbitol dehydrogenase, isocitrate dehydrogenase, alpha-ketoglutarate dehydrogenase, succinate dehydrogenase, malate dehydrogenase, yellow enzyme, glutamate dehydrogenase, glycerol 1-phosphate dehydrogenase, or a combination thereof.

7. The method of claim 6, wherein the third enzyme is glutamate dehydrogenase.

8. The method of claim 1, wherein substrate of the third enzyme is α-ketoglutarate.

9. The method of claim 1, wherein the fluorophore is selected from a group consisting of nicotinamide adenine dinucleotide (NAD⁺), NADH, nicotinamide adenine dinucleotide phosphate (NADP⁺), NADPH, flavin adenine dinucleotide (FAD), FADH₂, flavin mononucleotide (FMN), FMNH₂, Coenzyme A, Coenzyme Q, tryptophan tryptophylquinone (TTQ), pyrroloquinolinequinone (PQQ), adenosine triphosphate (ATP), guanosine triphosphate (GTP), cytidine triphosphate (CTP), thymidine triphosphate (TTP), uridine triphosphate (UTP), adenosine diphosphate (ADP), guanosine diphosphate (GDP), cytidine diphosphate (CDP), thymidine diphosphate (TDP), and uridine diphosphate (UDP), or any combination thereof.

10. The method of claim 9, wherein the fluorophore is NADPH.

11. The method of claim 1, wherein the decrease in fluorescence signaling is proportional to arginase-1 activity in the sample.

12. The method of claim 1, wherein the decrease in fluorescence signal comprises measuring fluorescence at a defined time interval over a defined incubation period to obtain a real-time kinetic fluorescent trace, wherein arginase-1 activity is calculated form the rate of decrease in fluorescence signal over time.

13. An enzyme couple assay system, the system comprising:
a first enzyme comprising arginase;
a first substrate;
a second enzyme to form ammonia; and
a third enzyme, a substrate of the third enzyme, and a fluorophore.

14. The system of claim 13, wherein the first enzyme is arginase I.

15. The system of claim 13, wherein the first substrate is L-arginine.

16. The system of claim 13, wherein the second enzyme is urease.

17. The system of claim 13, wherein the third enzyme is glutamate dehydrogenase, and the substrate of the third enzyme is α-ketoglutarate.

18. The system of claim 13, wherein the fluorophore is selected from a group consisting of nicotinamide adenine dinucleotide (NAD⁺), NADH, nicotinamide adenine dinucleotide phosphate (NADP⁺), NADPH, flavin adenine dinucleotide (FAD), FADH₂, flavin mononucleotide (FMN), FMNH₂, Coenzyme A, Coenzyme Q, tryptophan tryptophylquinone (TTQ), pyrroloquinolinequinone (PQQ), adenosine triphosphate (ATP), guanosine triphosphate (GTP), cytidine triphosphate (CTP), thymidine triphosphate (TTP), uridine triphosphate (UTP), adenosine diphosphate (ADP), guanosine diphosphate (GDP), cytidine diphosphate (CDP), thymidine diphosphate (TDP), and uridine diphosphate (UDP), or any combination thereof.

19. The system of claim 18, wherein the fluorophore is NADPH.

20. A kit comprising the assay system of claim 13.
